(19) United States
(12) Patent Application Publication
(10) Pub. No.: US 2007/0185514 A1
(43) Pub. Date: Aug. 9, 2007

(54) Microsurgical instrument

(76) Inventor: G. Lamar Kirchhevel, Laguna Niguel, CA (US)

Correspondence Address: ALCON, IP LEGAL, TB4-8, 6201 SOUTH FREEWAY, FORT WORTH, TX, 76134, US

(21) Appl. No.: 11/348,191

(22) Filed: Feb. 6, 2006

(51) Int. Cl. A61B 17/32

(52) U.S. Cl. 606/171

(57) ABSTRACT

A microsurgical instrument including a cutting member, a base, a nose member, and an actuating handle providing the ability to safely adjust an open size of a port of the cutting member while the instrument is cutting tissue.

### FIELD OF THE INVENTION

The present invention generally pertains to microsurgical instruments. More particularly, but not by way of limitation, the present invention pertains to microsurgical instruments having a port for aspirating and cutting tissue.

### DESCRIPTION OF THE RELATED ART

Many microsurgical procedures require precision cutting and/or removal of various body tissues. For example, certain ophthalmic surgical procedures require the cutting and/or removal of the vitreous humor, a transparent jelly-like material that fills the posterior segment of the eye. The vitreous humor, or vitreous, is composed of numerous microscopic fibers that are often attached to the retina. Therefore, cutting and removal of the vitreous must be done with great care to avoid traction on the retina, the separation of the retina from the choroid, a retinal tear, or, in the worst case, cutting and removal of the retina itself.

The use of microsurgical cutting probes in posterior segment ophthalmic surgery is well known. Such vitrectomy probes are typically inserted via an incision in the sclera near the pars plana. The surgeon may also insert other microsurgical instruments such as a fiber optic illuminator, an infusion cannula, or an aspiration probe during the posterior segment surgery. The surgeon performs the procedure while viewing the eye under a microscope.

Conventional vitrectomy probes typically include a hollow outer cutting member, a hollow inner cutting member arranged coaxially with and movably disposed within the hollow outer cutting member, and a port extending radially through the outer cutting member near the distal end thereof. Vitreous humor is aspirated into the open port, and the inner member is actuated, closing the port. Upon the closing of the port, cutting surfaces on both the inner and outer cutting members cooperate to cut the vitreous, and the cut vitreous is then aspirated away through the inner cutting member. U.S. Pat. Nos. 4,577,629 (Martinez); 5,019,035 (Missirlian et al.); 4,909,249 (Akkas et al.); 5,176,628 (Charles et al.); 5,047,008 (de Juan et al.); 4,696,298 (Higgins et al.); and 5,733,297 (Wang) all disclose various types of vitrectomy probes, and each of these patents is incorporated herein in its entirety by reference.

Conventional vitrectomy probes include “guillotine style” probes and rotational probes. A guillotine style probe has an inner cutting member that reciprocates along its longitudinal axis. A rotational probe has an inner cutting member that reciprocates around its longitudinal axis. In both types of probes, the inner cutting members are actuated using various methods. For example, the inner cutting member can be moved from the open port position to the closed port position by pneumatic pressure against a piston or diaphragm assembly that overcomes a mechanical spring. Upon removal of the pneumatic pressure, the spring returns the inner cutting member from the closed port position to the open port position. As another example, the inner cutting member can be moved from the open port position to the closed port position using a first source of pneumatic pressure, and then can be moved from the closed port position to the open port position using a second source of pneumatic pressure. As a further example, the inner cutting member can be electromechanically actuated between the open and closed port positions using a conventional rotating electric motor or a solenoid. U.S. Pat. No. 4,577,629 provides an example of a guillotine style, pneumatic piston/mechanical spring actuated probe. U.S. Pat. Nos. 4,909,249 and 5,019,035 disclose guillotine style, pneumatic diaphragm/mechanical spring actuated probes. U.S. Pat. No. 5,176,628 shows a rotational dual pneumatic drive probe.

Most conventional vitrectomy probes are sized to have a relatively large fully open port size (e.g. 0.020 inches to 0.030 inches) for use in a variety of surgical objectives. Operating at relatively low cut rates (e.g. up to 800 cuts/minute), these probes may be used to remove large amounts of vitreous in a single cut cycle, such as in core vitrectomy, and to cut physically large vitreous tissue, such as traction bands. In addition, these probes are also used to perform more delicate operations such as mobile tissue management (e.g. removing vitreous near a detached portion of the retina or a retinal tear), vitreous base dissection, and membrane removal. However, the combined effect of large port size, large cut stroke, and relatively slow cut rate of these probes sometimes creates unwanted turbulence of the vitreous and retinal tissues and a large peak to peak fluctuation of intraocular pressure within the eye. Both of these limitations cause difficulty for the surgeon and can be detrimental to the patient.

Specialized vitrectomy probes have been developed. For example, probes with relatively smaller fully open port sizes (e.g. 0.010 inches) have been used to perform more delicate surgical objectives near the retina. An example of such a specialized probe is the Microport® probe available from Alcon Laboratories, Inc. of Fort Worth, Tex. However, these probes are not highly effective for core vitrectomy, and thus the surgeon is often forced to use and repeatedly insert multiple vitrectomy probes within a patient's eye, complicating the surgery and increasing trauma to the patient. Relatively high cut rate probes have been developed by Storz Instrument Company of St. Louis (the “Lightning” probe) and Scieran Technologies, Inc. of Laguna Hills, Calif. (the “Vit Commander” probe). However, it is believed that these probes are somewhat limited in flow rate, rendering them less effective for core vitrectomy.

With many conventional vitrectomy probes, the inner cutting member is always actuated from a fully open port position, to a fully closed port position, and back to a fully open port position in each cut cycle. U.S. Pat. Nos. 4,909,249 and 5,019,035 disclose mechanical apparatus for adjusting the open port size of a vitrectomy probe comprising a adjustment nut on the proximal end of the probe. Adjustment of the open size of the port requires one hand to hold the body of the probe and a second hand to rotate the nut. Such adjustment is not practical or safe with the cutting tip of the probe disposed inside the eye. In addition, such adjustment does not allow a surgeon to visualize the amount of open port adjustment with the cutting tip outside the eye because the operating microscope and associated lighting is set up to view the inside of the eye.

U.S. Pat. Nos. 6,514,268 and 6,773,445 disclose methods of operating conventional vitrectomy probes to vary open port size via adjusting the duty cycle and cut rate of the probe using a foot controller. However, such a system is dependent on the pneumatic system used to drive the inner cutting member of the probe and is therefore subject to system pressure output variations.

Therefore, a need exists for an improved vitrectomy probe that performs all of the fundamental aspects of vitrectomy surgery (i.e. core vitrectomy, mobile tissue management, vitreous base dissection, and membrane removal) and does not suffer from the above-described limitations.

### SUMMARY OF THE INVENTION

In one aspect, the present invention is microsurgical instrument including a cutting member, a base, a nose member, and an actuating handle. The cutting member has a tubular outer cutting member with a port for receiving tissue and a tubular inner cutting member disposed within the outer cutting member. The base has an actuating mechanism for reciprocating actuation of the inner cutting member so that the inner cutting member opens and closes the port and cuts tissue disposed in the port. The nose member has a cam member for operative engagement with the inner cutting member. The actuating handle is coupled to the base and operatively engaged with the cam member. The actuating handle also has a plurality of flexible appendages disposed around the instrument. The flexible appendages are capable of elongation upon application of a radially inward pressure. During actuation of the inner cutting member and upon application of the pressure, the appendages elongate to rotate the cam member, the cam member interrupts a return stroke of the inner cutting member, and an open size of the port is adjusted.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a perspective view of a microsurgical instrument according to a preferred embodiment of the present invention;

FIG. 2 is a top view of the microsurgical instrument of FIG. 1;

FIG. 3 is a side, sectional view of the microsurgical instrument of FIG. 1 shown operatively coupled to a microsurgical system;

FIG. 4 is an enlarged, perspective view of the cam member of the microsurgical instrument of FIG. 1;

FIG. 5 is a cross-sectional view of the cam member of FIG. 4; and

FIG. 6 is an enlarged, fragmentary, side, sectional view of the portion of the microsurgical instrument of FIG. 1 shown in circle 6 of FIG. 2.

### DETAILED DESCRIPTION OF PREFERRED EMBODIMENTS

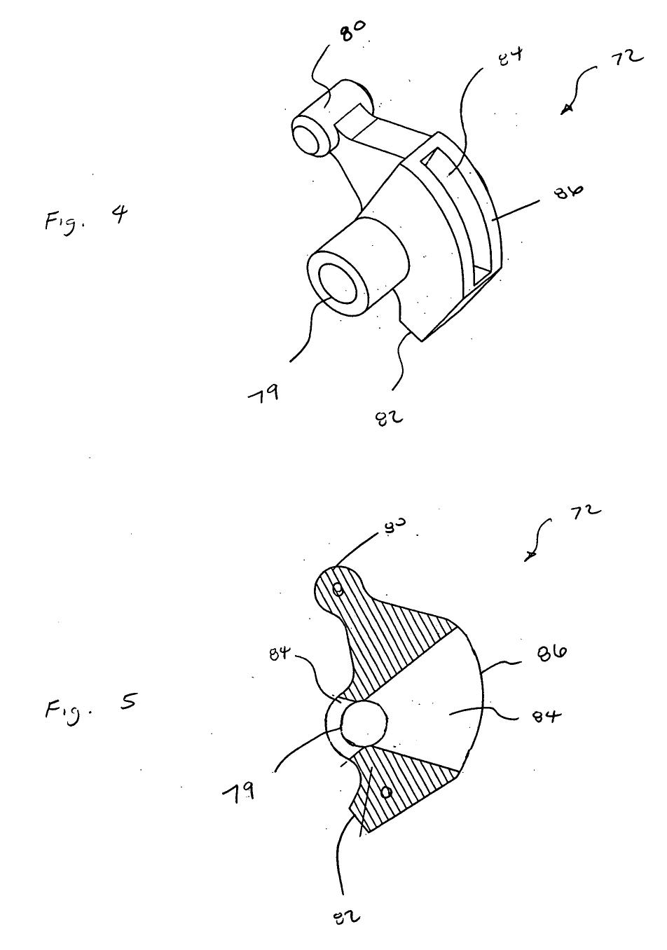

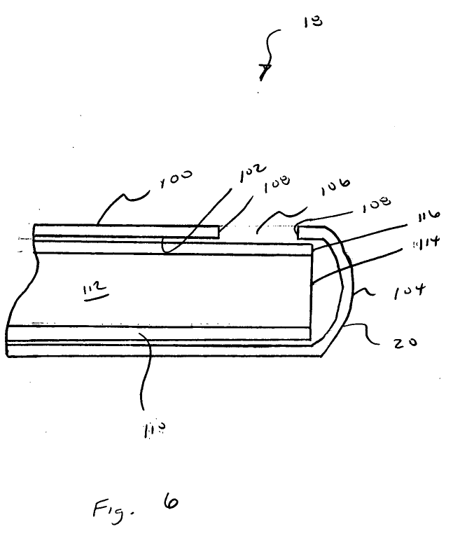

The preferred embodiments of the present invention and their advantages are best understood by referring to FIGS. 1 through 6 of the drawings, like numerals being used for like and corresponding parts of the various drawings.

Microsurgical instrument 10 generally includes a base 12, an actuating handle 14, a nose member 16, and a cutting member 18 having a distal tip 20. As shown in the Figures, microsurgical instrument 10 is a vitrectomy probe. However, microsurgical instrument 10 may be any microsurgical cutting, aspiration, or infusion probe.

Base 12 includes an actuating mechanism 13 for actuating a tubular inner cutting member 110 of cutting member 18 in a reciprocating manner. Actuating mechanism 13 preferably includes a first pneumatic port 22, a second pneumatic port 24, a diaphragm chamber 26, a flexible diaphragm 28, and a rigid center support 30. Flexible diaphragm 28 is frictionally coupled to center support 30 and base 12. Base 12 further includes an aspiration port 34 and a distal portion 12a having an aperture 12b and a distal tip 12c. A collar 36 couples distal portion 12a to actuating handle 14. Inner cutting member 110 is coupled to center support 30 and is slidably and fluidly coupled to base 12 via o-rings 38.

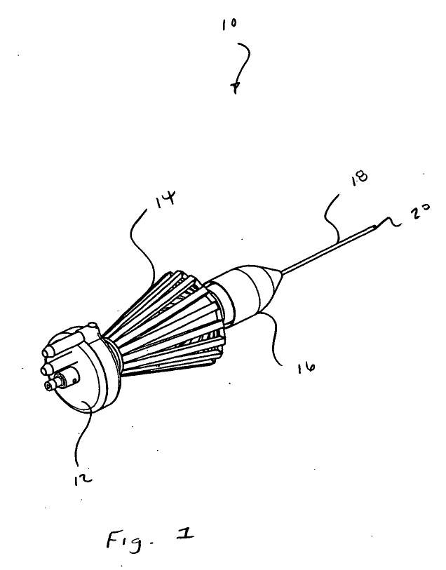

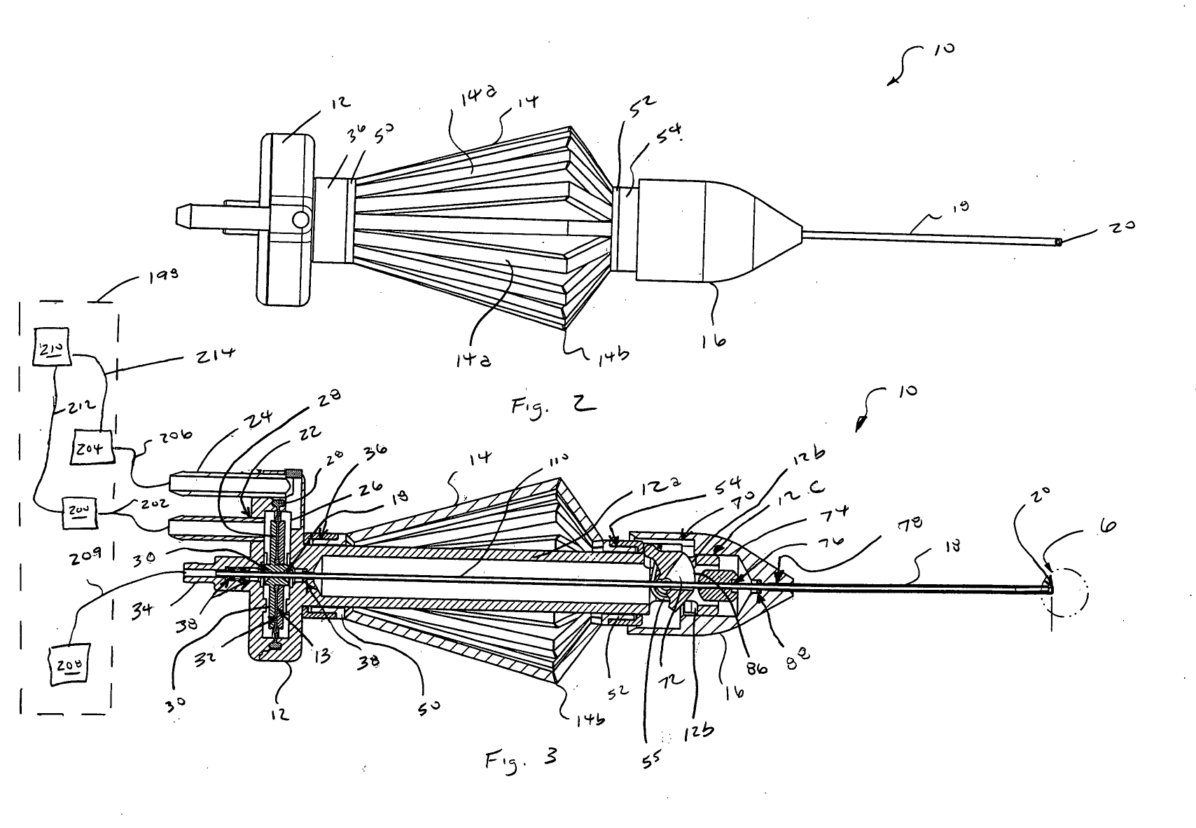

Actuating handle 14 preferably includes a proximal base 50, a distal base 52, and a plurality of flexible appendages 14a coupled to both bases 50 and 52. Flexible appendages 14a may be made from any suitable springy material having a memory, such as titanium, stainless steel, or a suitable thermoplastic. Handle 14 surrounds distal portion 12a of base 12. Proximal base 50 is coupled to collar 36. Distal base 52 is received within a slidable collar 54. A user grasps microsurgical instrument 10 via handle 14. When a user exerts an inward pressure on flexible appendages 14a, flexible appendages 14a bend at or near 14b, straightening and elongating flexible appendages 14a, and moving collar 54 toward distal tip 20. When such pressure is removed, spring 55 returns flexible appendages 14a to the position shown in FIG. 2.

Nose member 16 preferably includes cam chamber 70 for receiving a cam member 72, a base chamber 74 for receiving distal tip 12c of base 12, a bushing 76 for receiving inner cutting member 110 of cutting member 18, and an outlet 78 for receiving a tubular outer cutting member 100 of cutting member 18. Cam member 72 is rotationally coupled to nose member 16 within aperture 12b of base 12 via dowel pins (not shown) inserted into each end of a bore 79. Cam member 72 preferably has a first stopping surface 80 for interfacing with collar 54, a second stopping surface 82 for interfacing with base 12, a clearance slot 84 for receiving inner cutting member 110 of cutting member 18, and a cam surface 86 for interfacing with bushing 76. An o-ring 88 slidably and fluidly seals nose member 16 to inner cutting member 110.

As described above, cutting member 18 preferably includes tubular outer cutter member 100 and tubular inner cutting member 110. Outer cutting member 100 has an inner bore 102, a closed end 104, a port 106 for receiving tissue, and cutting surfaces 108. Inner cutting member 110 has an inner bore 112, an open end 114, and a cutting surface 116.

In operation, vitrectomy probe 10 is operatively coupled to a microsurgical system 198. More specifically, pneumatic port 22 is fluidly coupled to a pneumatic pressure source 200 via a fluid line 202, pneumatic port 24 is fluidly coupled to a pneumatic pressure source 204 via fluid line 206, and aspiration port 34 is fluidly coupled to vacuum source 208 via fluid line 209. Inner bore 112 and fluid line 209 are primed with a surgical fluid. Microsurgical system 198 also has a microprocessor or computer 210, which is electrically coupled to pneumatic pressure sources 200 and 204 via interfaces 212 and 214, respectively.

A surgeon inserts distal tip 20 into the posterior segment of the eye using a pars plana insertion. The surgeon selects a desired vacuum level for vacuum source 208. Tissue is aspirated into inner bore 112 via port 106. The surgeon selects a desired cut rate for probe 10 using microprocessor 210 and optionally a proportional control device (not shown), such as a foot controller. More specifically, microprocessor 210 uses pressurized gas sources 200 and 204 to create a cyclic pressure differential across diaphragm 28 so as to move center support 30, and thus inner cutting member 110, in a reciprocating manner at the desired cut rate. When the pressure provided to pneumatic port 22 is greater than the pressure provided to pneumatic port 24, inner cutting member 110 is moved toward distal tip 20 until open end 114 is past cutting surface 108, as shown in FIG. 6. This actuation closes port 106, allowing cutting surfaces 108 and 116 to cut the tissue within inner bore 112. The cut tissue is aspirated through inner bore 112, aspiration port 34, fluid line 209, and into a collection chamber (not shown). When the pressure provided to pneumatic port 24 is greater than the pressure provided to pneumatic port 22, inner cutting member 110 is moved away from distal tip 20, opening port 106 and allowing the further aspiration of tissue.

During actuation of inner cutting member 110, a user may exert pressure on flexible appendages 14a at or near 14b and to straighten and elongate appendages 14a. Collar 54 contacts first stopping surface 80, and cam member 72 rotates about bore 79 moving second stopping surface 82 toward base 12. As the user continues to straighten and elongate appendages 14a, cam surface 86 begins to contact bushing 76 on the return stroke of cutting member 110. Such contact interrupts the return stroke of inner cutting member 110 and decreases the open size of port 106 from its fully open size. Due to the changing radius of cam surface 86, additional straightening and elongation of appendages 14a causes further interruption of the return stroke of cutting member 110 and further decrease in the open size of port 106. When the user reduces or eliminates pressure on flexible appendages 14a, spring 55 rotates cam member 72 in the opposite direction, increasing the open size of port 106. The present invention thus allows the open port size of port 106 to be adjusted to any point between 100% (fully open) and 0% (fully closed) during operation of probe 10 and with distal tip 20 in the eye. The present invention correspondingly provides variable flow control through port 106 and inner bore 112 to accommodate different surgical objectives.

From the above, it may be appreciated that the present invention provides significant benefits over conventional vitrectomy probes. For example, the present invention allows for adjustment of open port size using one hand versus two hands, allows a surgeon to easily visualize the amount of open port adjustment via the operating microscope, and allows for the adjustment of open port size independent of the console settings of vacuum and cut rate, console pressure variations, or probe friction or tolerance variations. Most importantly, the present invention greatly increases the safety of cutting tissue near the retina by providing a surgeon significantly more control over open port size and flow rate.

The present invention is illustrated herein by example, and various modifications may be made by a person of ordinary skill in the art. For example, although the present invention is described above in connection with a vitrectomy probe, it is equally applicable to aspiration probes, infusion probes, and other cutting probes.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A microsurgical instrument, comprising:
a cutting member having a tubular outer cutting member with a port for receiving tissue and a tubular inner cutting member disposed within said outer cutting member;
a base having an actuating mechanism for reciprocating actuation of said inner cutting member so that said inner cutting member opens and closes said port and cuts tissue disposed in said port;
a nose member having a cam member for operative engagement with said inner cutting member; and
and an actuating handle coupled to said base and operatively engaged with said cam member, said actuating handle having a plurality of flexible appendages disposed around said instrument, said flexible appendages being capable of elongation upon application of a radially inward pressure;
wherein during actuation of said inner cutting member and upon application of said pressure, said appendages elongate to rotate said cam member, said cam member interrupts a return stroke of said inner cutting member, and an open size of said port is adjusted.

2. The microsurgical instrument of claim 1 wherein application of said pressure causes said open size of said port to decrease.

3. The microsurgical instrument of claim 2 further comprising a spring operatively engaged with said cam member, so that upon reduction or elimination of said pressure, said spring causes said cam member to rotate in an opposite direction, and said open size of said port increases.

4. The microsurgical instrument of claim 1 wherein a user may apply said pressure and adjust said open size of said port using a single hand that is also used to grip said instrument by said actuating handle.

5. The microsurgical instrument of claim 1 wherein said instrument is a vitrectomy probe.
